(19) United States
(12) Patent Application Publication
(10) Pub. No.: US 2003/0119976 A1
(43) Pub. Date: Jun. 26, 2003

(54) One-component isocyanate-crosslinking two-phase compositions

(76) Inventors: Otto Ganster, Odenthal (DE); Gerhard Klein, Monheim (DE); Jorg Buchner, Bergisch Gladbach (DE); Heinz-Werner Lucas, Bergisch Gladbach (DE); Anja Matussek, Dusseldorf (DE)

Correspondence Address: BAYER CORPORATION, PATENT DEPARTMENT, 100 BAYER ROAD, PITTSBURGH, PA, 15205, US

(21) Appl. No.: 10/217,237

(22) Filed: Aug. 12, 2002

(30) Foreign Application Priority Data
Aug. 16, 2001 (DE) 10140206.6

(51) Int. Cl. C07C 249/00; C08K 3/00

(52) U.S. Cl. 524/589; 560/330

(57) ABSTRACT

The present invention relates to surface-deactivated solid isocyanates obtained by surface reaction of finely dispersed solid isocyanates with mono- or polyamines that have anionic groups or groups capable of anion formation and have primary and/or secondary amino groups.

The present invention also relates to a process for the preparation of the solid, surface-deactivated isocyanates by dispersing finely divided, solid isocyanates in a liquid medium and reacting them with mono- or polyamines having primary and/or secondary amino groups and having anionic groups or groups capable of anion formation.

Finally, the present invention relates to compositions containing the solid, deactivated isocyanates according to the invention and isocyanate-reactive dispersions of homo- and co-polymers of olefinically unsaturated monomers and/or polyurethane dispersions.

### FIELD OF THE INVENTION

The present invention relates to aqueous dispersions of finely dispersed surface-deactivated solid isocyanates, and to compositions containing these dispersions and their use for the production of layers, films or powders of latent reactivity for adhesive compounds or coatings.

### BACKGROUND OF THE INVENTION

EP-A 0 204 970 describes a process for the preparation of stable dispersions of finely divided polyisocyanates by treatment of the polyisocyanates in a liquid with stabilizers and the action of high shear forces or milling. Suitable di- and polyisocyanates are those whose melting points are above 10° C., preferably above 40° C. For the production of the retarding or surface-deactivating polymer casing that surrounds the isocyanate particles, mono- or poly-functional amine stabilizers having primary and/or secondary amine groups are used. The resulting dispersions are used as crosslinkers.

EP-A 0 505 889 describes aqueous dispersions of encapsulated polyisocyanates, which are prepared by dispersion of the isocyanates in water and surface reaction with primary or secondary amines having a molecular weight below 400. The polyisocyanates can be used in unmodified or hydrophilically modified form.

EP-A 0 467 168 discloses aqueous preparations of copolymer dispersions and finely divided surface-deactivated polyisocyanate solid suspensions. They are used as coating agents for woven and nonwoven substrates. The deactivating agents are compounds that convert isocyanate groups located at the surface to urea or polyurea structures, such as water or primary and secondary amines. Crosslinking of the coatings produced using such preparations takes place at the same time as drying at elevated temperature.

EP-A 0 922 720 describes aqueous dispersions that contain a surface-deactivated solid polyisocyanate and an isocyanate-reactive polymer. The dispersions are used to produce storage-stable layers or powders of latent reactivity, which are made to crosslink by heating above an activation temperature. The preparation of the polyisocyanate dispersion and the surface deactivation are carried out according to EP-A 0 204 970.

WO-A 99/58590 also describes storage-stable, surface-deactivated, isocyanate-containing dispersion preparations, which as dried films, crosslink at temperatures below 70° C.

In the preceding prior art cited deactivation is carried out by reacting the exposed isocyanate groups at the surface of the solid isocyanate particles to form urea groups. It has been found that dispersions in water of such solid isocyanates (treated with mono- or poly-amines as deactivating agent) can also readily be stirred again after sedimentation, especially when longer-chain polyether amines, such as Jeffamine D 400 or Jeffamine T 403 (Huntsman Corp., Utah, USA), are used as the deactivating amine. However, a disadvantage of this type of surface deactivation is that preparations of isocyanates so stabilized in polymer dispersions, for example in polyurethane dispersions such as Dispercoll U 53 or U 54 (Bayer AG; Leverkusen; Germany), reduce the shear stability, and spray processing in particular is impaired by the formation of coagulate spots. Those two problems are more pronounced the greater the excess of deactivating amine not consumed in the surface reaction with the dispersed isocyanate particles.

An object of the present invention is to deactivate the particles of solid isocyanates at the surface such that the resulting preparations obtained therefrom with polymer dispersions have improved shear stability and the formation of coagulate spots is prevented.

It has now been found that the disadvantages of the prior art are overcome if mono- or poly-amines that have anionic groups or groups capable of anion formation and also have primary and/or secondary amino groups are used for surface deactivation. The ionic groups are anchored chemically to the surface of the polyisocyanate by reaction of the amino groups with the isocyanate groups to form urea groups. A stabilizing casing having anionic groups is thus produced for the polyisocyanate, which is otherwise unchanged.

### SUMMARY OF THE INVENTION

The present invention relates to surface-deactivated solid isocyanates obtained by surface reaction of finely dispersed solid isocyanates with mono- or polyamines that have anionic groups or groups capable of anion formation and have primary and/or secondary amino groups.

The present invention also relates to a process for the preparation of the solid, surface-deactivated isocyanates by dispersing finely divided, solid isocyanates in a liquid medium and reacting them with mono- or polyamines having primary and/or secondary amino groups and having anionic groups or groups capable of anion formation.

Finally, the present invention relates to compositions containing the solid, deactivated isocyanates according to the invention and isocyanate-reactive dispersions of homo- and co-polymers of olefinically unsaturated monomers and/or polyurethane dispersions.

It has been found that no negative effect on the resistance of the isocyanates with regard to the reaction with water can be ascertained as a result of the hydrophilic modification of the particles caused by the surface deactivation according to the invention.

### DETAILED DESCRIPTION OF THE INVENTION

Suitable solid isocyanates include di- and polyfunctional solid isocyanates, or mixtures thereof, having a melting point above 40° C., preferably above 80° C. Examples include diphenylmethane-4,4′-diisocyanate (4,4′-MDI), naphthalene-1,5-diisocyanate (NDI), 1,4-phenylene diisocyanate, dimeric 1-methyl-2,4-phenylene diisocyanate (dimer of 2,4-TDI), 3,3-diisocyanato-4,4′-dimethyl-N,N-diphenylurea (TDIH), and the isocyanurate of isophorone diisocyanate (IPDI). Preferred polyisocyanates are the dimer of 2,4-TDI, TDIH, and the isocyanurate (trimerization product) of IPDI. Dimeric 2,4-TDI is particularly preferred.

Suitable mono- and polyamines for the surface deactivation are those that have primary and/or secondary amino groups and have terminal or lateral, anionic groups or groups capable of anion formation, especially carboxylate and/or sulfonate groups, as a constituent of the molecular structure. Deactivation (or “stabilization”) of the solid isocyanate is carried out by reacting the deactivating agent with the exposed isocyanate groups at the surface of the solid isocyanate particles to form urea groups.

Suitable deactivating agents include the salts, especially alkali salts, of mono- or poly-aminosulfonic acids. Preferred are the salts of α-amino acids, such as glycine, lysine, glutamic acid and aspartic acid. Preferred salts of ω-amino acids have the formula I

H₂N—R—COO⁽⁻⁾ X⁽⁺⁾ (I)

wherein

R represents a hydrocarbon radical having 2 to 17 carbon atoms and

X⁽⁺⁾ represents an alkali metal cation or a substituted ammonium group.

Examples include the salts of aminopropionic acid (β-alanine), 4-aminobutyric acid and 6-aminohexanoic acid.

Also preferred are salts of diaminocarboxylic acids corresponding to formula II

H₂N—A—NH—B—COO⁽⁻⁾ X⁽⁺⁾ (II)

wherein

A and B are independently hydrocarbon radicals having 2 to 6 carbon atoms, preferably 2 carbon atoms, and

X⁽⁺⁾ represents an alkali cation or a substituted ammonium group.

Preferred aminosulfonates are diaminosulfonates corresponding to formula III

H₂N—A—NH—B—SO₃⁽⁻⁾X⁽⁺⁾ (III)

wherein A, B and X⁽⁺⁾ are as defined above.

A particularly preferred diaminosulfonate compound of formula III is the sodium salt of 2-(2-amino-ethylamino)-ethanesulfonic acid. The use of this salt as the deactivating agent results in low viscosities of the dispersions containing the solid, surface-deactivated isocyanates according to the invention. This represents a considerable advantage in terms of processing in the case of dispersion in bead mills, for example, because separation of the dispersion is substantially simpler to carry out than in the case the very pasty dispersions that are formed when non-ionic stabilizing amines are used.

The deactivation can be carried out in various ways:

a) By introducing the powdered solid isocyanate into a solution of the deactivating agent and dispersing it therein. The deactivating agent does not have to be completely in solution. Preferably, it is an aqueous solution or a solution in a liquid medium that is not a solvent for the isocyanate.

b) Low-melting polyisocyanates can be dispersed and deactivated by introducing the melt into a solution of the deactivating agent cooled below the solidification point of the isocyanate. Preferably, it is an aqueous solution or a solution in a liquid medium that is not a solvent for the isocyanate.

c) By adding the deactivating agent or a solution to the dispersion of the finely divided isocyanate in a liquid. The solvent and the dispersing medium are preferably water or a liquid medium that is not a solvent for the isocyanate.

Particle sizes of the solid isocyanates of less than 50 μm, preferably less than 20 μm and more preferably less than 10 μm are required for the surface-deactivated, solid isocyanates according to the invention. The required particle size is achieved by milling the solid isocyanates prior to dispersion and subsequent deactivation, or alternatively by combining the deactivating operation with the fine distribution by carrying out the dispersion using suitable milling and dispersing devices in the presence of the deactivating agent. Devices suitable for the fine dispersion include dissolvers, dispersing devices of the rotor-stator type, ball mills and bead mills, in which the temperature should not exceed 40° C. The dispersion of a melt of the isocyanate is also possible using jet dispersers.

The equivalent ratio of the amino groups to the total isocyanate groups present in the solid isocyanate is from 0.001 to 0.3, preferably 0.05 to 0.15 and more preferably 0.01 to 0.1.

The degree of deactivation of the isocyanate can be altered as desired, at the expense of the ability to subsequently activate the dry film of latent reactivity, by varying the indicated isocyanate/amine equivalent ratios upwards or downwards. As the amount of amine increases, the urea covering on the surface of the polyisocyanate particles becomes more dense and the deactivating casing becomes more stable.

In addition to the deactivating amine, the liquid, preferably aqueous, medium used for the deactivation and fine distribution of the polyisocyanate can contain emulsifiers, thickeners, protective colloids, stabilizers, antioxidants, fillers, pigments, plasticizers, non-solvent liquids and other known additives.

Suitable resins for preparing the compositions containing the solid isocyanates deactivated according to the invention are the known isocyanate-reactive aqueous dispersions of homo- and co-polymers of olefinically unsaturated monomers and/or polyurethane dispersions. The amount of deactivated solid isocyanate in the compositions according to the invention, based on amount of polymer, is 0.5 to 20 wt. %, preferably 2 to 10 wt. %, and more preferably 3 to 5 wt. %.

The compositions may also contain other known resins and additives such as dispersions that do not contain isocyanate-reactive groups, emulsifiers, thickeners, protective colloids, stabilizers, antioxidants, fillers, pigments, plasticizers and non-solvent liquids.

The compositions according to the invention preferably contain 20 to 99.9 wt. % of the isocyanate-reactive polymer dispersions, 0.1 to 13 wt. % of solid deactivated isocyanates, and 0 to 79.9 wt. % of additives, wherein the preceding percentages are based on the weight of the compositions.

Suitable polymers of olefinically unsaturated monomers are described, for example, in EP-A 0 206 059. They include homo- and copolymers based on acrylic acid esters of C₁ to C₁₈ alcohols, or homo- and copolymers based on vinyl esters of carboxylic acids having 2 to 18 carbon atoms, preferably 2 to 4 carbon atoms, such as vinyl acetate. They can optionally be used with up to 70 wt. %, based on the total amount, of other olefinically unsaturated monomers and/or homo- or copolymers of (meth)acrylic acid esters of alcohols having 1 to 18 carbon atoms, preferably 1 to 4 carbon atoms, such as the methyl, ethyl, propyl, hydroxyethyl or hydroxypropyl esters of (meth)acrylic acid.

Isocyanate-reactive groups are incorporated by copolymerization of OH- or NH-functional monomers, such as hydroxyethyl or hydroxypropyl (meth)acrylate, butanediol monoacrylate, ethoxylated or propoxylated (meth )acrylates, N-methylol-acrylamide, tert-butylamino-ethyl methacrylate or (meth)acrylic acid. Glycidyl methacrylate and allyl glycidyl ether can also be copolymerized. The subsequent reaction of the epoxy groups with amines or amino alcohols results in secondary amino groups.

Also suitable are aqueous dispersions of polymers or copolymers of 2-chloro-1,3-butadiene, optionally with the previously mentioned olefinically unsaturated monomers. These dispersions have a chlorine content of 30 to 40 wt. %, preferably 36 wt. %. The reactivity of the non-isocyanate-reactive polymers of 2-chlorobutadiene is obtained by the replacement, which takes place during the preparation process, of hydrolyzable Cl groups by OH groups, or according to EP-A 0 857 741. (Examples of polychloroprene dispersions having various degrees of hydrolysis, Table 1, page 5 with CR dispersions 1 to 4.)

Suitable aqueous polyurethane dispersions are those described, for example, in U.S. Pat. No. 3,479,310, U.S. Pat. No. 4,092,286, DE-A 2 651 505, U.S. Pat. No. 4,190,566, DE-A 2 732 131 or DE-A 2 811 148.

Preferred polymer dispersions are isocyanate-reactive polyurethane and/or polyurea dispersions, and polymers of 2-chlorobutadiene. Particularly preferred are dispersions of isocyanate-reactive polyurethanes containing crystallized polymer chains which, as measured by means of thermomechanical analysis, decrystallize at least partially at temperatures of +23° C. to +110° C., preferably +23° C. to +90° C. and more preferably +23° C. to +65° C.

When producing the preparations according to the invention it must be ensured that the dispersions of the solid, surface-deactivated isocyanates yield a homogeneous mixture with the polymer dispersions to ensure uniform distribution of the solid isocyanate content. That is achieved by the use of stirring and mixing units having a sufficiently high distributing action that are conventionally employed in industry.

The compositions according to the invention are distinguished over deactivation using non-ionic amines by very much better shear stability and especially by the prevention of the formation of coagulate spots, which impede spray processing. After drying there are obtained very uniform, visually homogeneous, smear-free and smooth films that have such high surface quality that they are suitable not only as adhesive layers but also for the production of optically demanding surface coatings.

Compositions containing the surface-deactivated solid isocyanates may also be used for the production of coatings having latent reactivity.

Latent reactivity means that possible crosslinking reactions of the polymer with the isocyanate do not occur either during preparation or in the dried coating. It is thus possible to produce storable preparations or coatings. Crosslinking is only initiated by brief heat activation, but then takes place in the course of several days at RT without the additional supply of heat. The coatings have a markedly increased softening point and resistance to water and solvents.

The compositions according to the invention may also be used to provide an adhesive bond of latent reactivity. The bonds are obtained by application of the compositions to either one side or both sides of the substrates to be bonded and subsequent drying. Activation may be achieved with the brief supply of heat and simultaneous joining.

For the production of these adhesive bonds, the adhesive coating, which has dried on the substrate to be bonded, is decrystallized by heating for a short time, preferably for 30 to 60 seconds, at temperatures of +65° C. to 110° C., and joined in the decrystallized state. That can be carried out by application to both sides or to one side. For the application of adhesive to one side, the preparation according to the invention is applied to a substrate and dried, and is then pressed with a film material that has been softened plastically by heating. As a result of the contact with the adhesive film, the latter assumes a temperature above the decrystallization temperature of the polymer and the heat activation is initiated.

Suitable substrates are all substrates that have adequate adhesion to the adhesive film. Examples include wood, pressed wood-fiber material, thermoplastic resins, thermosetting plastics, textiles and leather.

The compositions according to the invention may also be used to provide adhesive films of latent reactivity that are obtained by applying the compositions to a substrate, subsequent drying and removal of the substrate as a film.

Suitable substrates are those that do not have good adhesion to the adhesive film, so that the adhesive strip of latent reactivity can be removed without difficulty. Examples include polytetrafluoroethylene, silicone rubber, silicone-treated paper, and polished chromium or aluminium surfaces coated with release agent.

The compositions according to the invention may also be used to provide powders of latent reactivity that are obtained by spray drying the compositions according to the invention. The adhesive films and powders so produced can be stored at temperatures below the decrystallization temperature of the polymer and crosslink when heated above that limit, preferably at temperatures of +65° C. to +110° C.

It is also possible to process the compositions according to the invention by carrying out the crosslinking step and the drying step simultaneously. Temperatures of +60 to 110° C., preferably +80 to +110° C., are necessary therefore.

The following examples merely illustrate the invention. Those skilled in the art will recognize many variations that are within the spirit of the invention and scope of the claims.

### EXAMPLES

The following tests were prepared with the dimer of 1,4-toluylene diisocyanate (DesmodurTT/G, Rhein Chemie, Mannheim, particle size less than 50 μm; NCO content: 24.0%; m.p.: 156° C.).

The following substances were used in the examples:

BYK 028—antifoaming agent; manufacturer: BYK Chemie GmbH, D-46483 Wesel

Necal BX—emulsifier; manufacturer: BASF AG, D-67056 Ludwigshafen

Jeffamine D 400—stabilizing amine; manufacturer: Huntsman Corp., Utah, USA

isophorone diamine (IPDA)—stabilizing amine; manufacturer: Merck-Schuchard, D-85662 Hohenheim

sodium salt of 2-(2-amino-ethylamino)-ethanesulfonic acid—stabilizing amine; Bayer AG, D-0214 Leverkusen

Dispercoll U 53, polyurethane dispersion having a decrystallization temperature of approx. 55° C.; manufacturer: Bayer AG, D-0214 Leverkusen

Borchigel L 75—thickener; manufacturer: Borchers GmbH, D-40765 Monheim

I. Preparation of Deactivated Dispersions of Desmodur TT Dimer in Water (According to the Invention)

The amounts indicated in the basic formulation (Table 2) of water, antifoaming agent, emulsifier, and stabilizer amine (amounts: see Table 1) were placed in a bead mill together with 50 vol. % of glass beads (φ=3 mm), and a homogeneous mixture was produced by stirring. Desmodur TT dimer was then added in an amount of 300 g, and the mixture was dispersed for 20 minutes at 2000 rpm. The suspension was separated from the beads over a sieve. Since the suspension was not stable to sedimentation, homogenization was carried out again by stirring before partial amounts were removed.

The deactivation of Desmodur TT dimer was carried out with the following amines.

TABLE 1
The amounts of amine in g, calculated
on 300 g of DesmodurTT dimer.

| | | | Equiv. NCO/NH₂ | | |
|---|---|---|---|---|---|
| | | Eq. | a) | b) | c) |
| Example | Amine | Wt. | 100:1 | 100:3 | 100:7 |
| 1) Comparison | IPDA (g/300 g | 85 g | 1.5g | 4.4 g | 10.1 g |
| | TT) | | (1a) | (1b) | (1c) |
| 2) Comparison | Jeffamine D | 115 g | 2.0 g | 5.9 g | 13.7 g |
| | 400 resin | | (2a) | (2b) | (2c) |
| | (g/300 g TT) | | | | |
| 3) Example | aminosulfonate* | (95 g*) | — | — | — |
| (according to | salt in the | 211 g | 3.6 g | 10.9 g | 24.8 g |
| the invention) | form of a 45% | | (3a) | (3b) | (3c) |
| | solution | | | | |
| | (g/300 g TT) | | | | |

*45% solution of the sodium salt of 2-(2-amino-ethylamino)-ethanesulfonic acid in water.

TABLE 2
Basic formulation of the deactivated isocyanate dispersions.

| | | | wt. % amine | wt. % TT in |
|---|---|---|---|---|
| | | Parts by | in the | the |
| | | weight | preparation | preparation |
| Water | | 435 | — | — |
| Antifoam | BYK 028 | 1.2 | — | — |
| Emulsifier | Necal BX dry | 3.8 | — | — |
| Isocyanate | Desmodur TT | 300 | — | 40.5 |
| | dimer | | | |
| Σ total | | 740 | — | — |
| Comparison | IPDA | 1.5 | 0.20 | 40.5 |
| 1a | | | | |
| Comparison | IPDA | 4.4 | 0.59 | 40.3 |
| 1b | | | | |
| Comparison | IPDA | 10.1 | 1.3 | 40.0 |
| 1c | | | | |
| Comparison | Jeffamine D 400 | 2.0 | 0.26 | 40.4 |
| 2a | resin | | | |
| Comparison | Jeffamine D 400 | 5.9 | 0.79 | 40.2 |
| 2b | resin | | | |
| Comparison | Jeffamine D 400 | 13.7 | 1.8 | 39.8 |
| 2c | resin | | | |
| Example 3a | Aminosulfonate* | 3.6 | 0.22 | 40.3 |
| Example 3b | salt solution, | 10.9 | 0.65 | 40.0 |
| Example 3c | 45% | 24.8 | 1.5 | 39.2 |

*45% solution of the sodium salt of 2-(2-amino-ethylamino)- ethanesulfonic acid in water.

II.) Production of the Adhesive Compositions Using a Polyurethane Dispersion

II.1) Formulation Without Thickening

100 parts by weight of Dispercoll U 53 polyurethane dispersion were placed in a vessel, and 10 parts by weight of the deactivated Desmodur TT dimer suspensions were added with stirring by means of a dissolver. The deactivated suspensions of Desmodur TT dimer all contained approximately 40 wt. % of solid isocyanate, which corresponded to 4.0 parts by weight of TT. For the purposes of homogenization, the mixtures were stirred for 5 minutes at 1000 rpm.

II.2) Formulation With Thickening

3 ml of Borchigel L 75 (20%) were then added as thickener by means of a single-trip pipette, and mixing was carried out for a further one minute at 1000 rpm. The viscosities of the mixtures were then 3800 to 13,200 mPa.s, according to the mixture.

III.) Viscosity Stability of the Adhesive Compositions

The viscosity stability during storage of the composition was an important technical parameter for reproducible processing. For that reason it was generally subject to closely formulated specification limits.

TABLE 3
Viscosity stability of the thickened adhesive compositions.

| | Viscosities of the adhesive compositions (mPa*s) | | | | | | | | |
|---|---|---|---|---|---|---|---|---|---|
| Days | 1a | 1b | 1c | 2a | 2b | 2c | 3a | 3b | 3c |
| Immediate | 3840 | 4200 | 6800 | 13200 | 12000 | 6980 | 4210 | 4400 | 6100 |
| 2 | 4100 | 4040 | 6050 | 10800 | 8050 | 6350 | 4210 | 3310 | 5940 |
| 7 | 3760 | 4070 | 6050 | 6310 | 7210 | 5180 | 4170 | 3220 | 5540 |
| 14 | 3670 | 3970 | 7100 | 6950 | 7680 | 6550 | 4070 | 3060 | 5270 |
| 21 | 3560 | 3940 | 6080 | 5300 | 7300 | 6550 | 4100 | 3090 | 5270 |

It can clearly be seen from Table 3 that the compositions containing solid, deactivated isocyanate according to the invention (see Table 2; 3a to 3c) have good viscosity stability, which was markedly better than that achieved with Jeffamine D 400 resin (Comparison Example 2a-2c). Accordingly, the compositions containing the solid isocyanate deactivated according to the invention meet the practical demands of storability with largely unchanged rheological properties.

IV.) Coagulate Formation During Storage of the Adhesive Composition

In particular during spraying, the adhesive compositions must be free of coagulated particles in order to ensure problem-free processing. This requirement is essential for the technical applicability of the adhesive compositions. In addition, the formation of coagulate spots during storage gives an indication of the shear stability of the formulation. This manifests itself in the resistance of the formulation to the stresses caused by stirring, mixing, and shaking during transportation.

TABLE 4
Unthickened adhesive compositions:

| | Coagulate spots | | | | | | | | |
|---|---|---|---|---|---|---|---|---|---|
| Days | 1a | 1b | 1c | 2a | 2b | 2c | 3a | 3b | 3c |
| Immediate | 0 | 1 | 1 | 1 | 2 | 2 | 0 | 0 | 0 |
| 4 | 2 | 2 | 2 | 1 | 2 | 2 | 0 | 0 | 0 |
| 7 | 3 | 3 | 3 | 3 | 3 | 3 | 0 | 0 | 0 |
| 12 | — | — | — | — | — | — | 1 | 0 | 0 |
| 30 | — | — | — | — | — | — | 3 | 2 | 0 |

Rating: 0 = no; 1 = few; 2 = pronounced; 3 = very pronounced

Of the compositions produced without the addition of a thickener, only the examples deactivated with the sodium salt of 2-(2-amino-ethylamino)-ethanesulfonic acid (see Table 4, Examples 3a to 3c according to the invention), with a sufficient concentration of the stabilizing amine (3a), formed no coagulate spots after storage for 30 days. In the case of Comparison Examples 1a-1c and 2a-2c, the coagulate spots form immediately in the unthickened compositions and increase from day to day. On only the fourth day, the amount was so great that the mixtures were unusable in practice.

TABLE 5
Thickened adhesive compositions:

| | Coagulate spots | | | | | | | | |
|---|---|---|---|---|---|---|---|---|---|
| Days | 1a | 1b | 1c | 2a | 2b | 2c | 3a | 3b | 3c |
| Immediate | 0 | 0 | 0 | 0 | 1 | 1 | 0 | 0 | 0 |
| 2 | 0 | 0 | 2 | 0 | 1 | 3 | 0 | 0 | 0 |
| 7 | 0 | 1 | 2 | 2 | 3 | — | 0 | 0 | 0 |
| 30 | 1 | 2 | 3 | 3 | — | — | 0 | 0 | 0 |

Rating: 0 = no; 1 = few; 2 = pronounced; 3 = very pronounced

The thickened compositions were more stable, but increased formation of coagulate spots was observed in Comparison Examples 1a-1c and 2a-2c (Table 5) after some time. Only compositions 3a to 3c according to the invention remained spot-free. If those compositions according to the invention were applied to a smooth substrate, very uniform layers with a smooth surface were obtained; whereas, the comparison examples prepared with amines that were not in accordance with the invention exhibited very uneven, rough surfaces due to the high content of spots.

V.) Effect of the Deactivating Amine on the Shear Stability of an Adhesive Composition

200 g of Dispercoll U 53 polyurethane dispersion were placed in a vessel and mixed for 2 minutes and with 20 g of isocyanate dispersion in a Dispermat mixer at 1000 rpm. Approximately 6 ml of Borchigel L 75 thickener (20% solution in water) were then added, and the formulation was stirred for a further 120 minutes at 1000 rpm. Samples were removed after 30, 60 and 120 minutes and were spread onto glass plates. The assessment of the coagulate formation of the individual formulations is shown in Table 6.

TABLE 6
Coagulate formation under shear load:

| Duration of the shear | Coagulate formation after x minutes | | | | | | | | |
|---|---|---|---|---|---|---|---|---|---|
| load in minutes | 1a | 1b | 1c | 2a | 2b | 2c | 3a | 3b | 3c |
| 30 | 1 | 3 | 3 | 3 | 3 | 3 | 0 | 0 | 0 |
| 60 | 3 | — | — | — | — | — | 0 | 0 | 0 |
| 120 | — | — | — | — | — | — | 0 | 0 | 0 |

Rating: 0 = no; 1 = few; 2 = pronounced; 3 = very pronounced

Because of ionically modified surface of the Desmodur® TT dimer particles according to the invention, a dispersion was formed that was substantially more shear stable than in the case of Comparison Examples 1c and 2c, and that showed no signs of coagulate formation even after an extreme shear load (120 minutes). To the contrary the comparison examples were coagulated after only 60 minutes in the most favorable case.

Thermostability of the Adhesive Bond After Shock Activation

The adhesive compositions produced according to II.2 (formulation with thickening) were tested immediately after their production and after storage for 4 weeks at RT.

Preparation of the Samples

The test specimens of Nora rubber (SBR) were roughened with abrasive paper (grain=80) immediately before application of the adhesive The adhesive formulation was applied by means of a brush to both sides of the adherend, which was 20×10 mm in size. The adhesive layer was dried for 60 minutes at 23° C./50% relative humidity.

Shock Activation

The adherends were irradiated for 10 seconds with an IR radiator from Funk (shock activation device 2000). Activation of the adhesive film on the NORA sample for 10 seconds gave a surface temperature of 115° C. The decrystallization temperature of the polymer chain of the polyurethane dispersion used (Dispercoll® U 54) was 55° C. Bonding took place immediately after heat activation of the adhesive-coated test specimens by placing the activated adhesive layers together and pressing them for one minute at 4 bar in a press. The test specimens so prepared were stored for 7 days at 23° C. and 50% relative humidity.

Heat Test

The test specimens were subjected to a 4 kg load and tempered at 40° C. for 30 minutes in a heating chamber. The test specimens were then heated to 150° C. at a linear heating rate of 0.5° C./minute. The softening point, i.e., the temperature in ° C. at which the bond fails under the 4 kg load, was recorded. 5 individual measurements were carried out in each case.

TABLE 7
Results on SBR (NORA rubber) as substrate using the freshly
produced adhesive compositions (immediate values) and the
adhesive compositions stored for 4 weeks at RT.

| Storage | | | | | | | | | |
|---|---|---|---|---|---|---|---|---|---|
| of the | | | | | | | | | |
| Com- | | | | | | | | | |
| position | Softening point (° C.)* | | | | | | | | |
| Weeks | 1a | 1b | 1c | 2a | 2b | 2c | 3a | 3b | 3c |
| Im- | 143 | 140 | 142 | 133 | 140 | 140 | 142 | 138 | 140 |
| mediate | | | | | | | | | |

| | | | | | |
|---|---|---|---|---|---|
| 4 weeks | Adhesive has | Adhesive has | 138 | 134 | 136 |
| | coagulated | coagulated | | | |

*Dispercoll U 53 polyurethane dispersion has a softening point of approximately 60° C. when bonding was carried out in the same manner without crosslinking.

This test shows that the thermostability of the adhesives according to the invention yielded good results after bonding both immediately and after storage for four weeks.

Although the invention has been described in detail in the foregoing for the purpose of illustration, it is to be understood that such detail is solely for that purpose and that variations can be made therein by those skilled in the art without departing from the spirit and scope of the invention except as it may be limited by the claims.

What is claimed is:

1. Surface-deactivated solid isocyanates obtained by surface reaction of finely dispersed solid isocyanates with a mono- or polyamine having anionic groups or groups capable of anion formation and having primary and/or secondary amino groups.

2. The surface-deactivated solid isocyanates of claim 1 wherein the mono- or poly-amine has terminal or lateral, anionic groups or groups capable of anion formation as a constituent of the molecular structure.

3. The surface-deactivated solid isocyanates of claim 1 wherein the mono- or polyamine comprises a diaminocarboxylic acid corresponding to the formula
H₂N—A—NH—B—COO⁽⁻⁾ X⁽⁺⁾ (II)
wherein
A and B are independently hydrocarbon radicals having 2 to 6 carbon atoms and
X⁽⁺⁾ represents an alkali cation or a substituted ammonium group.

4. The surface-deactivated solid isocyanates of claim 1 wherein the mono- or polyamine comprises a diaminosulfonate corresponding to the formula
H₂N—A—NH—B—SO₃⁽⁻⁾X⁽⁺⁾ (III)
wherein
A and B are independently hydrocarbon radicals having 2 to 6 carbon atoms and
X⁽⁺⁾ represents an alkali cation or a substituted ammonium group.

5. The surface-deactivated solid isocyanates of claim 1 wherein the mono- or polyamine comprises the sodium salt of 2-(2-amino-ethylamino)-ethanesulfonic acid.

6. A process for the preparation of the surface-deactivated solid isocyanates of claim I which comprises dispersing finely divided solid isocyanates in a liquid medium and reacting them with a mono- or poly-amine having primary and/or secondary amino groups and having anionic groups or groups capable of anion formation.

7. The process of claim 6 wherein the ratio of the amino groups to the total isocyanate groups present in the solid isocyanate is 0.001:1 to 0.3:1.

8. A composition containing the surface-deactivated solid isocyanates of claim 1 and an isocyanate-reactive dispersion of a homopolymer or copolymer of one or more olefinically unsaturated monomers and/or a polyurethane dispersion.

9. The composition of claim 8 wherein a polyurethane dispersion is present in which the polyurethane is composed of crystallized polymer chains which decrystallize, as determined by thermomechanical analysis, at least partially at a temperature of +23° C. to +110° C.

10. An adhesive bond having latent reactivity that is obtained by applying the composition of claim 8 to either one side or both sides of the substrates to be bonded, subsequent drying the adhesive bond and activating the bond with a heat.

11. An adhesive film having latent reactivity that is obtained by applying the composition of claim 8 to a substrate, subsequent drying the bond and removing the substrate as a film.

12. A powder having latent reactivity that is obtained by spray drying the composition of claim 8.

13. A coated substrate obtained by applying a composition comprising the surface-deactivated solid isocyanates of claim 1 to a substrate.
